(19) United States
(12) Patent Application Publication
(10) Pub. No.: US 2025/0017716 A1
(43) Pub. Date: Jan. 16, 2025

(54) IMPROVED TEXTILE GRAFTS

(71) Applicant: Diaxamed LLC, Greenville, SC (US)

(72) Inventors: Aaron Hjelle, Greenville, SC (US); Robert Thatcher, Blaine, MN (US); Brian McMurray, Pinebluff, NC (US); Stephanie Norris, Rocky Point, NC (US); Joseph M. Shepley, Wernersville, PA (US); Savannah R. Forrester, Mauldin, SC (US); Lindsey Sanders Calcutt, Greenville, SC (US)

(21) Appl. No.: 18/833,705

(22) PCT Filed: Mar. 28, 2023
(86) PCT No.: PCT/US2023/065052

§ 371 Date: Jul. 26, 2024

Related U.S. Application Data
(60) Provisional application No. 63/362,082, filed on Mar. 29, 2022.

(51) Int. Cl. A61F 2/06; A61L 27/54; A61L 27/56

(52) U.S. Cl. CPC A61F 2/06; A61L 27/54; A61L 27/56; A61F 2210/0076; A61F 2250/0069; A61F 2250/0076; A61F 2250/0098

(57) ABSTRACT

Textile-based implant or prosthetic tubular or non-tubular products are provided that require two materials, textile and an impermeable, non-organic hydrophobic or super hydrophobic material, that when constructed into a graft product of the present invention produce three distinct zones. The resulting self-sealing implant or prosthetic product is super anti-wetting and therefore provides for easier explantation from the body. Explantation is further improved by the resistance of one surface to electrosurgical energy such as electrocautery. The implant or prosthetic products also prevent leakage as well as transmural communication or migration through the graft body or wall while still allowing ingrowth of tissue along at least a portion of one surface of the implant or prosthetic for secured anchoring.

### CROSS-REFERENCE TO RELATED APPLICATIONS

This application claims priority to U.S. provisional application No. 63/362,082, filed Mar. 29, 2022 and titled TEXTILE GRAFTS, the entire contents of which is incorporated herein by reference.

### STATEMENT REGARDING FEDERALLY SPONSORED RESEARCH OR DEVELOPMENT

Not Applicable

### BACKGROUND OF THE INVENTION

### Field of the Invention

The invention relates to textile products generally. More specifically, textile-based grafts that are stable and anchor after implantation, do not allow transmural communication or migration through the graft and that have improved anti-wetting properties on one surface that prevents bodily incorporation and provides easier explantation.

### Description of the Related Art

The art generally describes layered grafts comprising a textile with a focus on vascular grafts that are self-sealing to aid in repeated punctures in AV grafts for dialysis patients. Some known layered grafts comprise one surface covered by semi-impermeable, non-porous, water insoluble sealing material with limited anti-wetting properties and, generally initially, a removable masking material or coating on an opposing surface, e.g., an inner luminal surface of a tubular graft, of the textile's interstices and/or pores to prevent the water insoluble material from seeping or migrating through the textile and ultimately preventing tissue ingrowth that is necessary for implantation stability.

Certain known grafts comprise three layers of material, with the self-sealing material sandwiched between an inner and an outer layer. For example, the Triplex™ vascular graft commercialized by Vascutek Terumo comprises an inner layer of uncoated woven Dacron graft and an outer layer of expanded polytetrafluoroethylene (ePTFE). These two layers are fused together by a middle or central layer of self-sealing elastomeric membrane which is the thermoplastic elastomer Styrene-Ethylene-Propylene-Styrene, an organic (styrene) copolymer. The Triplex™ graft has been shown to have a water leakage value of 0.68 mL/cm²/min which provides good, but incomplete, water (or blood) impermeability. See De Paulis, et. al., A Third Generation of Ascending Aorta Dacron Graft: Preliminary Experience, Ann Thorac Surg 2008; 85:305-09. It is noted that De Paulis et al., state that the three layers confer total impermeability. However, the measured water leakage value reported by De Paulis et al., is not 0, but is instead 0.68 mL/cm²/min. The three-layer construction of Triplex™ cannot, therefore, be described as impermeable.

A modification of the typical three-layered vascular graft is illustrated by a two-layered vascular graft commercialized by Vascutek Terumo, commercially known as Biplex™ and which provides an inner uncoated woven polyester layer that is coated on the outer tubular surface with a self-sealing elastomer that is the same thermoplastic elastomer styrene copolymer used in the middle Triplex™ layer. For the same reasons described above regarding the Triplex™ construction and related non-zero water leakage value, Biplex™ also cannot accurately be described as completely water, or blood, impermeable.

In addition, neither of the described Vascutek Terumo vascular graft products discussed above can be described as “non-porous”. In other words, both Biplex™ and Triplex™ will allow transmural migration of cells and other materials from one side of the graft wall to and through the other side of the graft wall after implantation. The transmural communication has generally been considered to be a requirement for effective long-term implanting of, e.g., vascular grafts, whereby the transmural communication leads to tissue ingrowth on both sides of the graft, e.g., on both luminal and abluminal surfaces for an exemplary vascular or tubular graft, which results in bodily incorporation of the graft by the host tissues.

The result high level of bodily incorporation of the graft does provide anchoring stability, but it also greatly increases the difficulty of explanting the graft from the ingrown tissue, both in terms of actually visualizing the graft but also in terms of separating the graft from surrounding tissue which typically requires electrocautery. Known grafts fail to present outer layers that are impervious to electrocautery or that do not allow electrocautery energy to penetrate through the graft's outer layer to damage the textile layer.

One object of the present invention is, therefore, to provide a graft having an surface and/or layer that is impervious to energy applied to the graft by electrosurgical devices, for example and without limitation, electrocautery energy.

Another object of the present invention is to provide a graft with a sealing layer that protects an underlying textile layer from exposure to and/or damage from electrocautery energy.

Another object of the present invention is to provide a graft with a sealing layer that minimizes or mitigates damage to an underlying textile layer from electrocautery energy.

Another object of the present invention is to provide a graft with a sealing layer that minimizes, mitigates, or prevents damage to an underlying textile layer from electrocautery energy at low power, medium power, or high power.

Another object of the present invention is to provide a graft that has a water leakage value, or water permeability, that within the range of 0.68 mL/cm²/min and 0 mL/cm²/min at 120 mm Hg pressure. Preferably the water leakage value, or water permeability, is less than 0.16 mL/cm²/min at 120 mm Hg pressure. Most preferably, the water leakage value, or water permeability, will be substantially 0 mL/cm²/min at 120 mm Hg pressure.

Another object of the present invention is to prevent transmural communication or migration of cells or materials from one side or surface of the graft to and/or through the other side or surface of the graft.

Another object of the present invention is to prevent full and complete bodily incorporation of the implanted graft on the sealing agent side or surface of the graft structure.

Another object of the present invention is to enable or promote partial bodily incorporation of the implanted graft, wherein the incorporation portion(s) are easily accessed and separated from bodily tissues.

Another object of the present invention is to allow biological tissue and/or platelets to adhere, and stimulate tissue ingrowth, on at least part of one side or surface of the graft, while preventing total biological tissue and/or platelet adhesion or ingrowth on the other side or opposing surface of the graft.

Another object of the present invention is to allow biological tissue and/or platelets to adhere, and stimulate tissue ingrowth, on at least part of one side or surface of the graft, while promoting or enabling biological tissue and/or platelet adhesion or ingrowth only on selected portions of the other side or opposing surface of the graft.

Moreover, generally, the outer coating of known vascular grafts comprise organic polymers with carbon-to-carbon bonds that have a binding energy of 355 KJ/mol. As a result, these organic polymeric outer layers will provide limited resistance to electrocautery procedures which are typically required during explantation of the implanted graft.

Therefore, another object of the present invention is to provide a sealing layer and surface of the graft that is more resistant to electrocautery than sealing layers formed from known organic polymeric materials.

Another object is to provide a sealing layer that retards, slows and/or minimizes penetration and/or damage during electrocautery procedures at all power and voltage levels.

Another object is to provide a sealing layer that protects the underlying fabric layer from penetration and/or damage during electrocautery procedures at all power and voltage levels.

Still another object is to provide a sealing layer that protects the underlying fabric layer by retarding, slow, and/or minimizing penetration of electrocautery energy through the sealing layer at all power and voltage levels.

Another object is to provide an outer sealing layer that is impervious to electrocautery at all power and voltage levels.

Another object of the present invention is to provide an outer sealing layer that comprises a completely, or partially, smooth outer or external surface to discourage attachment of biological tissue and/or platelets thereto.

Another object of the present invention is to provide an outer sealing layer that protects against adherence of bacteria thereto and that protects against infection developing between the outer sealing layer and host tissue.

Still another object of the present invention is to protect against and/or prevent seroma formation between the sealing agent and the host tissue.

Another object of the present invention is to incorporate antibiotic materials or compounds on, or in, the outer sealing layer.

Known vascular grafts also are difficult to visualize during implant and explant procedures when blood is present. Therefore, providing an outer sealing layer, or one surface, of a graft that is hydrophobic to super hydrophobic and/or anti-wetting to super anti-wetting as a further object of the present invention. In this manner, blood is shed away from the outer scaling layer of the inventive embodiments, making it easier for the practitioner to see the implanted device.

Another object of the present invention is to provide certain embodiments with a colorant along at least a portion of the outer sealing layer, wherein the colorant contrasts with blood and tissue.

As discussed above, known layered grafts require a removable mask or coating applied over an exemplary inner luminal surface of the textile of a tubular graft to prevent sealing agent from migrating through the textile layer to reach the luminal surface, which will prevent tissue ingrowth at that surface, thus reducing anchoring stability upon implant. This approach reduces the thickness of the finished scaling layer, reduces the stiffness and increases the flexibility of the graft product, and requires using a removable mask that effectively blocks migration of the sealing agent to ensure that at least part of the inner luminal surface is not covered by the sealing agent and that sufficient scaling agent remains on an opposing or outer surface to provide the necessary protective functionality.

Known layered grafts using a removable mask or coating to protect a surface, e.g., an inner or luminal surface, of a graft from migration of the sealing agent to the inner or luminal surface all require the removable mask or coating to be placed on the luminal surface, e.g., of a vascular graft, then the mask or coating is removed to reveal underlying textile without scaling agent therein. This arrangement allows the sealing agent to migrate substantially through the inner or textile layer, thus leaving a relatively small distance that is untouched by sealing agent as a result of the masking or coating protection. In turn, a reduced amount of the width or thickness of an inner or textile layer is available for tissue ingrowth.

It is therefore another object of the invention to reserve most of the thickness of the inner or textile layer for tissue ingrowth.

Known layered grafts also require that the mask or coating applied to the inner luminal surface be removed by active, e.g., etching, or passive, e.g., water solubility, prior to implantation.

The present invention addresses, inter alia, these issues.

### BRIEF SUMMARY OF THE INVENTION

Textile-based graft products are provided that require two materials, a textile and a sealing agent. In some embodiments, the sealing agent is an impermeable, non-organic hydrophobic or super hydrophobic material, that when constructed into a graft product of the present invention produce three separate cross-sectional zones. The resulting self-sealing graft product is super anti-wetting and therefore provides for easier explantation from the body. Explantation is further improved by the resistance of the sealing agent to electrocautery. The graft product also prevents blood leakage as well as transmural communication or migration through the graft body or wall while still allowing ingrowth of tissue along at least a portion of one surface of the graft for secured anchoring within the body.

### BRIEF DESCRIPTION OF THE SEVERAL VIEWS OF THE DRAWINGS

Embodiments of the invention will now be described, by way of example, with reference to the following Figures.

FIGS. 1A and 1B illustrate end perspectives of one embodiment of the present invention.

FIG. 2 illustrates an end or cross-sectional view of one embodiment of the present invention.

FIG. 3A illustrates a cross-sectional view of one embodiment of the present invention.

FIG. 3B illustrates a side view of one embodiment of the present invention.

FIG. 3C illustrates a side view of one embodiment of the present invention.

FIG. 4A illustrates a perspective view of one embodiment of the present invention.

FIG. 4B illustrates a perspective view of one embodiment of the present invention.

FIG. 4C illustrates a perspective view of one embodiment of the present invention.

FIG. 4D illustrates a perspective view of one embodiment of the present invention.

FIG. 5 illustrates a top view of one embodiment of the present invention.

FIG. 6 illustrates a perspective view of one embodiment of the present invention.

FIG. 7 illustrates a cross-sectional view of one embodiment of the present invention.

FIG. 8 illustrates a side cutaway view of one embodiment of the present invention.

FIG. 9 illustrates a illustrates a side cutaway view of one embodiment of the present invention.

FIG. 10 illustrates a top view of one embodiment of the present invention.

### DETAILED DESCRIPTION OF THE INVENTION

Generally, various embodiments of the present invention are directed to devices and methods for a prosthesis or an implant, e.g., a graft such as a vascular graft, sheets, tapes and/or a patch, which may comprise a conduit or tubular portion. An exemplary graft may be cylindrical as well as a variety of other functional shapes, some of which are described herein. However, various embodiments are directed to non-tubular and/or non-conduit structures, such as flexible sheets, planar structures, tapes, or preformed structures that may be amenable to covering a non-tubular structure that may be flat or may comprise contours. Such non-tubular grafts may be used as patches, for organ repair, in pericardial procedures and the like as the skilled artisan will recognize. The prosthesis or implant, e.g., a graft or a patch embodiments described herein may comprise a textile which may further comprise a fabric, fiber or yarn and which may be woven and/or knitted or otherwise combined in a pattern or patterns to form the textile. Other configurations and materials for such a textile are known in the art and, as such, are within the scope of the present invention. Generally, the descriptions below may describe a particular embodiment of the prosthesis or implant as, e.g., a graft. However, as now made clear, this is not a limiting description as the inventive prosthetics and/or implants comprise all other embodiments described herein.

Embodiments of the present invention are directed to making an explantation procedure of the exemplary prosthesis or implant, e.g., a graft from a living body an easier process. Generally, an outer, or first, sealing layer that is not damaged by energy applied by, e.g., electrocautery is provided. In other embodiments, the outer, or first, sealing layer prevents the exemplary electrocautery energy from penetrating through the outer, or first, sealing layer to an inner, or second, textile layer, thus preventing damage to the inner, or second, textile layer.

In addition, the outer, or first, sealing layer may shed blood to make visualization during explantation or removal easier, so the outer, or first, sealing surface may be hydrophobic, or may be super-hydrophobic and/or super-antiwetting. In some embodiments, the outer, or first, sealing layer may be non-porous and have a surface roughness that aids in preventing biological tissue and/or platelet adherence, while an inner luminal, or second opposing, textile surface allows biological tissue and/or platelets to adhere thereto.

In some embodiments, the outer, or first, sealing layer may comprise a color that contrasts with blood and tissue to aid in visualizing during explantation or removal.

In some embodiments, the graft may, largely due to the outer, or first sealing layer's impermeability, comprise a water leakage or water permeability of substantially zero.

In some embodiments, the first, or outer sealing surface, prevents ingrowth of biological tissue into any portion of the first, or outer sealing surface, while at least a portion of the inner luminal, or second opposing, textile surface, allows biological tissue and/or platelets to adhere thereto and to grow into at least a portion of an inner, or second opposing, textile layer to provide anchoring stability within the body.

In some embodiments, transmural communication of biological materials, or water, from one side of the exemplary graft's wall to the other side of the graft's wall is prevented.

In some embodiments, the first, or outer sealing surface, prevents adherence of bacteria thereto, thereby preventing infection from occurring between the first, or outer, sealing surface and the host tissue that is in contact with the first, or outer sealing surface.

In some embodiments, the sealing agent is configured to prevent formation of seromas between the host tissue and the first, or outer, surface of the sealing layer.

In some embodiments, an antibiotic is taken up into, is embedded on, or covers, at least a portion of the scaling layer prior to implantation.

In some embodiments, an antibiotic is impregnated within a portion of the sealing layer.

In some embodiments, an antibiotic may be encapsulated and embedded within, or on, the sealing layer, wherein the capsule is configured to be biodegradable or dissolvable.

In some embodiments, a foaming agent may be combined with a polymer or co-polymer of an outer, or first, sealing layer to form a flexible structure that also comprises improved self-scaling capabilities.

In some embodiments, suture holes may self-seal to provide hemostasis within 10 minutes or less, within 5 minutes or less, within 2 minutes or less, within 1 minute or less, within 30 seconds or less, or within 10 seconds or less of suture hole creation.

In some embodiments, a dielectric polymer, or dielectric co-polymer, may comprise an outer, or first, sealing layer to form an electrocautery resistant graft.

In some embodiments radiopaque material, or radiopaque marker(s) are adhered to, embedded or impregnated within, and/or exposed within the sealing layer.

In some embodiments, two materials forming three cross-sectional zones may comprise the exemplary prosthesis or implant, e.g., a graft, wherein a first zone consisting only of an exemplary woven (or knitted) textile, a second zone consisting of a combination of the woven textile with a sealing agent having some or all of the properties described above, and a third zone consisting only of the scaling agent.

In some embodiments, the three-zoned embodiment is achieved without requiring a removable mask or coating to achieve the first zone consisting only of an exemplary woven or knitted textile.

In other embodiments, a removable mask or coating may be employed on an inner, or second opposing, surface of the exemplary woven or knitted textile to preserve a portion of the woven or knitted textile without contamination by the sealing agent of zone 3 as it migrates through the woven or knitted textile to create zone 2.

In some embodiments, one or more structures, regions or materials are provided on an external surface of the sealing layer to facilitate formation of a pseudo-intima on the luminal, or second, surface of the exemplary implant or prosthesis, e.g., graft.

In some embodiments, one or more structures, regions or materials are provided on an external surface of the sealing layer to enable controlled communication of biological material and/or cells through the sealing layer to facilitate tissue ingrowth on the textile-only surface.

In some embodiments, one or more structures, regions or materials are provided on an external surface of the sealing layer to enable controlled communication of biological material and/or cells through the sealing layer to facilitate anchoring stabilization by fibrotic incorporation with at least the external or outer surface of the sealing layer.

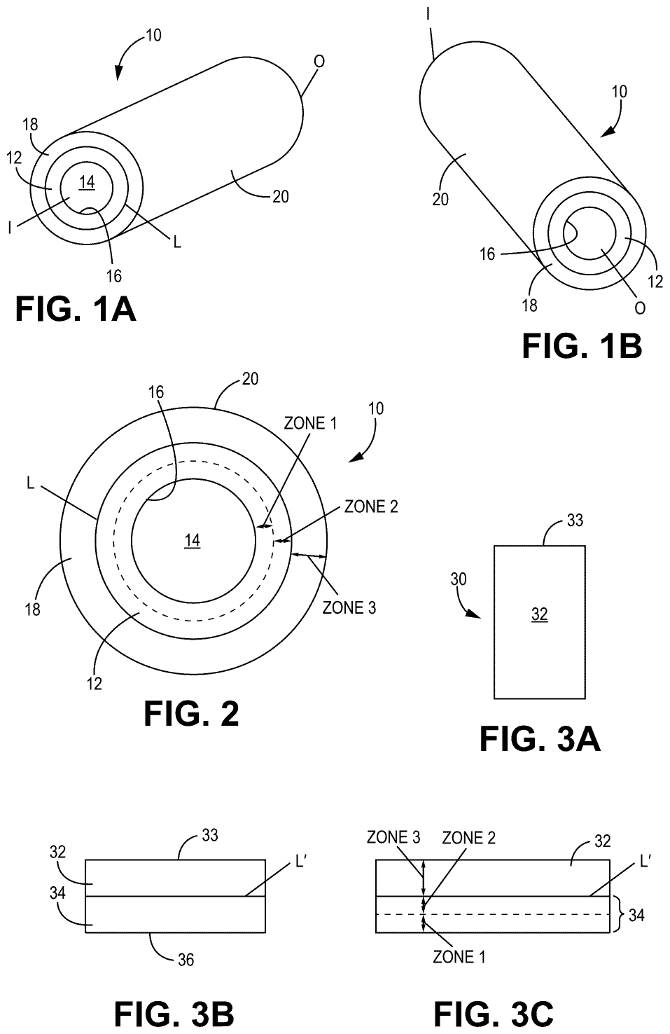

FIGS. 1A and 1B provide perspective view of one embodiment of an exemplary tubular graft or conduit graft or vascular graft. The exemplary tubular graft 10 comprises an inner textile layer 12 comprising a thickness of the textile layer 12 and defining a lumen 14 and an inner or luminal surface 16. Outer sealing layer 18 surrounds the inner layer 12, defining a thickness of the sealing layer 18 and comprising an outer or abluminal surface 20.

The illustrated graft 10 comprises an inlet I and an outlet O at opposing ends. However, it is understood that one or more inlets or outlets may be provided in alternative embodiments. Further, the graft may comprise the illustrated cylindrical shape or may comprise non-cylindrical shapes such as having one or more regions with larger outer diameters than other regions, multiple channels, a Y-shape or a T-shape, or a bulbous shape that may be used, e.g., for a Valsalva aortic root graft.

Inner textile layer 12 may comprise a textile that may be woven, braided, or knitted as the skilled artisan will readily appreciate. Accordingly, a plurality of interstices, spaces and/or pores are provided between the fiber, yarn and/or fabric of the textile. Some of these interstices, spaces and/or pores may allow adherence of biological tissue and/or platelets after implantation, followed by tissue ingrowth, thus anchoring and stabilizing the implanted graft.

### Electrocautery Resistance

Applicant has discovered that molecular binding energy of non-organic polymers such as silicone or silicone foam rubber is highly advantageous in that it is virtually impervious to energy applied by electrosurgical techniques and/or devices such as electrocautery energy. Because the molecular binding energy of organic polymers is lower, such materials are not impervious to electrocautery energy.

Electrocautery as used herein refers to a well-known process by which current is passed between an electrode and a cathode and may be monopolar or bipolar. The device may be applied to living tissue to achieve varying degrees of tissue destruction. In the case of explantation of a graft from a living body, tissue may overgrow portions or all of the graft surface(s). In the case of an exemplary tubular graft, electrocautery may be required to remove overlying tissue to allow visualization. It is critical to protect the exemplary graft from damage by the electrocautery heat energy. The recitation of electrocautery and eye-cautery are merely exemplary. The various embodiments herein are protective from any applied electrosurgical energy.

This is to be distinguished from eye-cautery devices and procedures which comprises a resistive wire through which current is passed. The resistive wire is heated and may be used to pierce through implanted or other grafts for various purposes. As a result, some embodiments of the tubular or non-tubular grafts described herein may enable eyecautery piercing through the sealing layer as well as the underlying textile layer. In this way, the graft embodiments are enabled to prevent damage to the underlying textile layer under all power and/or voltage settings of known electrocautery devices and as will be further described below, but also allow the eyecautery device to pierce or otherwise cut through the sealing layer, e.g., silicone as well as through the underlying textile graft.

### Working Example 1

Objective: To test the hypothesis that improved resistance over organic polymeric scaling agents is realized using non-organic polymeric scaling agent.

Materials and Methods: The experiment evaluated and compared (1) the Gelweave™ tubular graft commercialized by Acutek Terumo, composed of a woven textile (Dacron) that is coated with gelatin, an organic polymeric scaling agent; with (2) a woven textile graft coated with a layer of silicone.

The two tubular grafts were both pre-soaked in 0.9% saline and then tightly wrapped with uncooked chicken to simulate the conditions following implantation of the graft within a living body, wherein bodily tissue has wrapped around or incorporated the graft, thus requiring electrocautery to cut through the bodily tissue to reveal the graft for explanatation or inspection. The electrocautery device was Valleylab Force FX-C. User's Guide Force FX™-C Electrosurgical Generator with Instant Response™ Technology, copyright 2009, is hereby incorporated by reference in its entirety and is located at: 1023200.book (equippedmd.com)

The cautery parameters were set to 50% power, pure and cut mode.

Discussion: The Gelweave™ graft is found to be very reactive to the electrocautery energy. The pre-soaking saline permeates the graft, providing a low resistance electrical pathway. The organic polymer gelatin (scaling agent) provides little to no protection to the inner woven textile (Dacron), resulting in damage to the textile portion of the graft.

The textile graft covered by silicone was found to retard or slow any damage to the sealing layer and, in fact the sealing layer was observed to be completely non-reactive, i.e., completely impervious, to the electrocautery energy. As a result, the textile tubular layer was completely unaffected and undamaged by the electrocautery.

Conclusion: A non-organic polymer sealing agent the overlays or surrounds a textile layer slows or retards damage due to electrocautery energy. With the cautery parameters in this Example, the sealing layer is impervious to the electrocautery energy and/or protects the textile layer from electrocautery energy damage.

### Working Example 2

Objective: To test the hypothesis that non-organic polymeric sealing agent surrounding or covering a woven textile graft protects the woven textile graft from electrocautery damage by at least retarding and/or slowing any damage due to the electrocautery energy.

Materials and Methods: The experiment evaluated a woven textile tubular graft coated with a layer of silicone. This is the same type of woven textile graft discussed in Working Example 1.

The tubular graft was pre-soaked in 0.9% saline. The electrocautery device was also the same as used in Working Example 1: Valleylab Force FX-C.

In this series of experiments, the electrocautery device was engaged in both cut mode and coag mode at increasing power modes, all in mono-polar mode.

### Results:

TABLE 1
Electrocautery Device in Cut Mode

| | 25 watts | 50 watts | 75 watts | 100 watts | 200 watts | 300 watts |
|---|---|---|---|---|---|---|
| Pure | 1 | 1 | 1 | 1 | 1 | 1 |
| | 2 | 2 | 2 | 2 | 2 | 2 |
| Low | 1 | 1 | 1 | 1 | 1 | 1 |
| | 2 | 2 | 2 | 2 | 2 | 2 |
| Blend | 1 | 1 | 1 | 1 | 1 | N/A |
| | 2 | 2 | 2 | 2 | 2 | |

Sealing Layer and Textile Layer Protected; Damage to Sealing Layer and Textile Layer
1—Retarded;
2—No Penetration into Silicone; No Damage to Woven Textile

TABLE 2
Electrocautery Device in Coag Mode

| | 30 watts | 60 watts | 90 watts | 120 watts |
|---|---|---|---|---|
| Dessicate | 1 | 1 | 1 | 1 |
| | 2 | 2 | 2 | 2 |
| Fulgarate | 1 | 1 | 1 | 1 |
| | 2 | 2 | 2 | 2 |
| Spray | 1 | 1 | 1 | 1 |
| | 2 | 2 | 2 | 2 |

In addition, Working Example 2 included saline fluid within the tubular graft, clamped at both ends to retain the fluid therein during the electrocautery testing. The temperature of the saline was tested before, and then again after, the electrocautery testing. The temperature of the saline within the graft did not measurably increase after the electrocautery testing and, therefore, the saline fluid within the graft was unaffected in terms of heat increase by the electrocautery test treatments.

Thus, a non-organic polymer sealing agent, e.g., silicone, is an excellent heat insulator and electrical insulator. These characteristics allow the sealing layer to function to minimize and/or mitigate heat transfer and/or electrical conduction to and/or through the underlying textile layer. Provision of protection against heat transfer is significant. Blood passing through the graft is protected from thermal damage, e.g., hemolysis. In addition, the tissue that has grown into at least part of the textile layer, and//or portions of the sealing layer, is protected from thermal damage. And, in certain procedures, blood flow will be temporarily stopped, i.e., will be static, within the graft, making the blood cells within the graft and particularly those in closest proximity to the textile layer's luminal surface, vulnerable to thermal damage. Still further, aspects of the inventive grafts may comprise therapeutic cells and/or associated therapeutic agents and/or antibiotics or other substances adhered to or incorporated within the graft. The sealing layer discussed herein provides protection against heat transfer and resulting thermal damage to blood cells and ingrown tissue as well as therapeutic cells and any associated therapeutic agents and/or antibiotics or other substances adhered to or incorporated within the graft.

In each of Working Examples 1 and 2, silicone is also a strong dielectric, thus preventing a path to ground which further increases the resistance to electrocautery.

### Overall Conclusions Working Examples 1 and 2

Accordingly, the graft as described above comprises a sealing layer that:
(1) protects an underlying textile layer from damage from electrocautery energy in the tested device when set at all tested power levels, including the highest power levels, in both cut and coag modes;
(2) slows, retards, minimizes and/or at least partially mitigates damage to the sealing layer itself;
(3) slows, retards, minimizes and/or at least partially mitigates damage to an underlying textile layer from electrocautery energy at all tested power levels, including the highest power levels, in both cut and coag modes;
(4) provides at least some protection for an underlying textile from damage from electrocautery energy all tested power levels, including the highest power levels, in both cut and coag modes;
(5) provides protection against full and/or partial penetration into and/or through an underlying textile layer by electrocautery energy at all tested power levels, including the highest power levels;
(6) slows, retards protects against, and/or is not damaged by, electrocautery energy at all tested power levels, including the highest power levels, in both cut and coag modes; and
(7) provides heat transfer resistance and/or thermal protection for blood cells, ingrown tissue, therapeutic cells, agents antibiotics, substances, fluids, tissue, etc., located within the textile layer and/or on the side of the graft opposing the sealing layer that is receiving energy from electrocautery treatment.

These conclusions will apply equally to both tubular and non-tubular grafts having a non-organic polymer sealing layer or coating such as silicone.

Further, the exemplary non-organic polymeric sealing agent silicone has a higher binding energy than that of carbon (C—C) bonds (433 KJ/mol vs 355 KJ/mol) due to its siloxane bonds (—Si—O—Si—) forming the backbone of silicone (dimethyl polysiloxane). For the same reason, generally, inorganic polymeric sealing agents comprise a higher binding energy than organic polymeric sealing agents. This characteristic gives non-organic polymeric sealing agents a higher heat resistance than organic polymeric sealing agents. In turn, non-organic polymeric sealing agents thus have a higher resistance to electrocautery than organic polymeric sealing agent. Silicone is an exemplary non-organic polymer comprising added advantages in that it is, as discussed above, a dielectric material further increasing its resistance to electrocautery, is water impermeable and is non-porous.

FIG. 2 illustrates a cross-sectional view of one embodiment of the present invention wherein two materials form an exemplary tubular graft 10 comprising three zones composed of, or formed by, two materials as shown in cross section. Thus, the inner, or textile, layer 12 comprising a textile as described above is surrounded by the outer, or sealing, layer 18 formed from a scaling agent that, among other things as described herein, slows, retards and/or prevents electrocautery from reaching the inner textile layer 12. The concepts discussed herein apply to the subject prosthetics or implants generally.

As shown, innermost zone 1 consists only of the exemplary textile material. Zone 1 comprises a thickness that is less than the thickness of the textile layer 12. This is due to the sealing agent migrating radially inwardly to occupy a portion of the textile layer 12 by filling interstices and/or pores in the textile layer 12. Consequently, zone 2 has a combination of both the textile material and the sealing agent and has a thickness that is also less than the thickness of the textile layer 12. Zone 3 consists only of the sealing agent and comprises a thickness that may be equal to the thickness of the outer sealing layer 18, or that may be less than the sealing layer's 18 thickness due to loss of sealing agent material during the migration process to form zone 2. Thus, the nominal outer diameter of the tubular stent will reduce over time as the sealing agent moves inwardly and radially into the adjacent textile layer 12. Alternatively, scaling agent may be allowed to penetrate or migrate all the way through the exemplary textile, thus effectively eliminating zone 1. Other embodiments may comprise sealing agent penetrating through to at least part of the inner luminal surface 16, leaving the other regions of luminal surface 16 as textile only.

Zone 1 is bounded on an inner, luminal side by inner luminal surface 16.

Zone 2 is bounded on the outer side by interfacing surface L of the textile layer 12, wherein the sealing agent of sealing layer 18 at least initially interfaces with the textile layer 12 at the interface surface L.

The transition between zones 1 and 2 is shown as a dashed line in FIG. 2, representing the endpoint of the sealing agent's radially inward migration path across the interfacing surface L into the textile layer 12. As will now be apparent, the inner textile layer 12 is, following scaling agent migration, broken into the inner zone 1 and zone 2, wherein zone 2 at least partially surrounds zone 1.

As illustrated in FIGS. 3A-3C, planar grafts, sheet grafts, tapes, or other non-tubular shapes are also within the scope of the present invention. As discussed herein, such non-tubular shaped grafts may be used as patches, for organ repair, in pericardial procedures and other procedures as the skilled artisan will readily recognize. Exemplary planar graft 30 comprises a first or sealing layer 32 formed by a sealing agent that, as described herein and among other things, confers imperviousness to electrocautery in certain embodiments, the sealing layer 32 defining a first surface 33. A second, or textile, layer 34 is disposed next to the sealing layer 32 and is formed by the exemplary textile material described herein, the textile layer 34 defining a second surface 36. The line L′ illustrates the interfacing surface of the textile layer 34, and marking an at least initial boundary between the sealing and textile layers 32, 34. In alternate embodiments, and similar to the tubular graft embodiment of FIG. 2, three zones may be formed using the two materials of the first and second layers 32, 34 as shown in FIG. 3C.

Zone 1 consists only of the exemplary textile material.

Zone 2 consists of a mixture of the exemplary textile material and the scaling agent.

Zone 3 consists only of the sealing agent.

Consistent with the embodiment of FIG. 2, zone 1 comprises a thickness that is less than the thickness of the textile layer 34 due to the encroaching migration of the sealing agent of sealing layer 32 into the textile material.

Zone 2 comprises a thickness that extends from the interfacing surface L′ of textile layer 34 to the dashed line indicating the endpoint of the sealing agent's migration toward, and within, the textile material of textile layer 34 and toward the second surface 36. As shown, the thickness of zones 1 and 2 are both less than the thickness of the textile layer 34 as the textile layer 34 is formed from zone 1 and zone 2, wherein zone 2 is adjacent to zone 1.

Zone 3's thickness may be the same as the thickness of the sealing layer 32, and in some embodiments may become less thick than an initial thickness of the sealing layer 32 when initially applied to the textile layer due to the loss of sealing agent material to the migration process from sealing layer 32 to textile layer 34. It is highly preferred that zone 3 is provided, i.e., that the sealing agent does not all migrate to create zone 2, and that zone 3 comprises sealing agent defining a thickness for zone 3.

The embodiments of FIGS. 2 and 3A-3B each are illustrated as comprising a continuous boundary between zones 1 and 2, marking an endpoint of the migration of sealing agent into the textile layer of each embodiment.

Among other things, this creates a strong connection between the sealing layer and the textile layer of the exemplary tubular and non-tubular grafts 10, 30. In both embodiments, the sealing agent is prevented from migrating all the way to the inner luminal surface 16 in the tubular embodiment 10, and from reaching the second surface 36 of the non-tubular embodiment 30, thus providing uncontaminated textile along all of the surface area of those uncontaminated locations 16, 36 for tissue ingrowth upon implantation.

This result may be achieved by applying a mask or coating to the entire inner luminal surface 16 or the entire second surface 36 of the textile in order to prevent sealing agent from migrating thereto as disclosed in U.S. Pat. No. 10,926,003, the contents of which are incorporated by reference in its entirety.

Alternatively some sealing agent may be allowed to reach the inner luminal surface 16 of the tubular embodiment, or the second surface 36 of the non-tubular embodiment. As also disclosed in U.S. Pat. No. 10,926,003, this result may be achieved by selectively applying a mask or coating to the inner luminal surface 16 or to the second surface 36 in order to selectively prevent sealing agent from migrating to selected areas of the inner luminal surface 16 or second surface 36 of the textile, while allowing sealing agent to selectively migrate to other areas of the luminal surface 16 or second surface 36 of the textile.

According to U.S. Pat. No. 10,926,003, full migration prevention of the sealing agent, the removable mask or coating to the inner luminal surface of a tubular graft is achieved by application to cover the entire inner luminal surface 16 of a tubular graft. Further, U.S. Pat. No. 10,926,003 teaches that some sealing agent may be allowed to migrate to the inner luminal surface of a tubular graft by placing an exemplary mandrel inside the tubular graft's lumen, wherein the mandrel comprises apertures therethrough. The removable mask or coating is applied to the inner lumen of the mandrel, such that the applied removable mask or coating is applied radially outwardly through the apertures of the mandrel and to coat corresponding regions of the inner luminal surface 16 of the textile tube. In addition to teaching application of the mask or coating to the inner luminal surface 16, the removable mask or coating is also taught as requiring an active process such as etching or similar active, as opposed to a passive process such as water solubility, to remove the applied mask or coating.

An alternative mechanism to provide migration of some sealing agent to selected portions of the inner luminal surface 16 of a tubular graft, or to a second surface 36 of a non-tubular graft, comprises providing a mask or coating to selected portions of the interfacing surface L or L′.

Limited, but Effective, Bodily Incorporation of Sealing Layer with Easy Identification and Removal of Tissue Ingrowth Regions.

It is desirable to provide surface area of the sealing layer's outer surface with a material that does not promote tissue ingrowth. However, in some embodiments, portions or regions of the sealing layer's outer surface may be configured to promote tissue ingrowth for stability of the implanted graft.

Some embodiments may comprise at least one scrim region that may comprise fabric, fiber and/or yarn, or other non-sealing agent material embedded or incorporated along the outer surface of the sealing layer. In some embodiments, tissue growth promoting and/or antibiotic and/or immunosuppressive substances may be coated on the scrim or adhered to the scrim. Exemplary substances that may be coated or adhered to or seeded within the scrim are discussed below and comprise antibiotics, therapeutic cells, regulatory agents, immunosuppressive agents and the like.

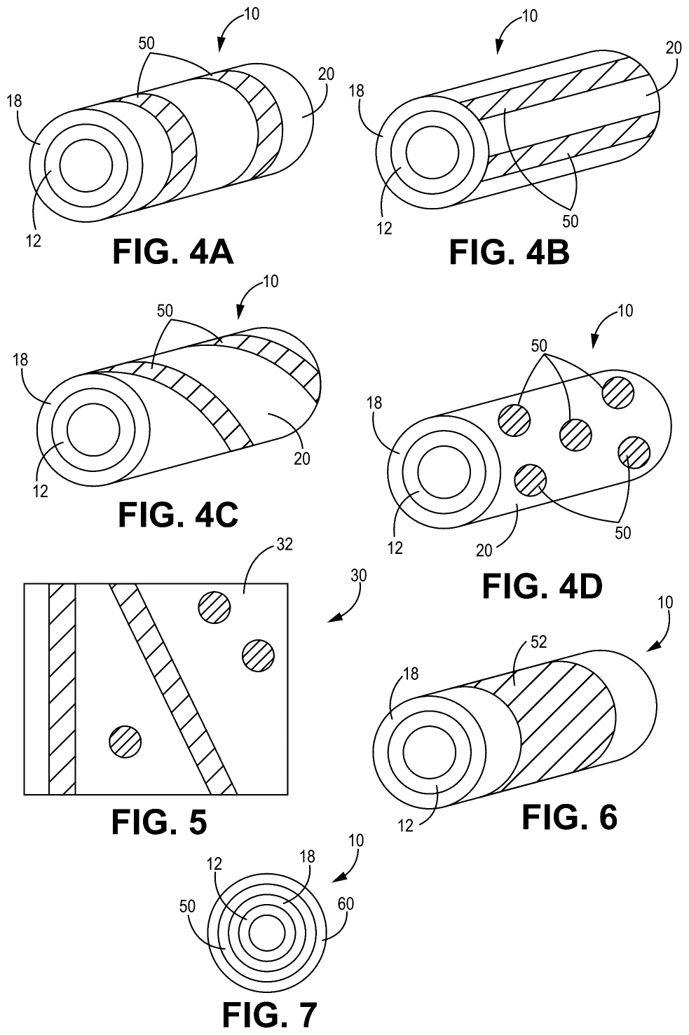

As seen in FIGS. 4A-4D, and with continued reference to FIGS. 1A-3C, exemplary scrims 50 may be circumferential (in the case of a tubular graft) or longitudinal, spiral, or may be applied randomly or in a pattern in order to cover a portion of the outer surface of the sealing layer 18 of the exemplary tubular graft 10 while providing additional surface area that does not promote tissue ingrowth. Exemplary scrims 50 may be applied to a tubular or to a non-tubular graft. See FIG. 5 for exemplary scrim 50 configurations in the sealing layer 32 of an exemplary non-tubular graft 30.

Generally, the scrim 50 may be embedded within the sealing layer 18 or 32 so as to not appreciably increase the outer diameter of the tubular or non-tubular graft 10, 30. In some of these embodiments, the scrim 50 may be applied to the interfacing surface L or L′ such that the scrim and the textile layer are in contact, without intervening sealing agent, and wherein the scrim is effectively surrounded by sealing agent. In other embodiments, the scrim may be adhered to the outer surface of sealing layer of the tubular or non-tubular graft 10, 30 and may slightly increase the outer diameter of the graft 10, 30 as a result.

Other embodiments may comprise a fabric, fiber, yarn and/or non-scaling agent material that may be knitted or woven or otherwise compiled and that may cover, or sleeve, at least a portion of the outer sealing layer of an implanted tubular or non-tubular graft. These solutions may also comprise antibiotics and/or tissue-growth promoting substances as described above. FIG. 6 provides an exemplary embodiment of a tubular exemplary graft 10 comprising a fabric sleeve 52 partially covering, and attached and/or adhered to the sealing layer 18. The fabric sleeve 52 may be in some embodiments, water soluble or otherwise biodegradable.

In some embodiments, the scrim and/or the fabric, fiber, yarn and/or non-scaling agent material covering or sleeve may comprise a color such as blue, or other color that provides a contrast with the tissue and blood to aid in locating the implanted graft and scrim and/or fabric covering or sleeve.

The scrim and/or the fabric, fiber, yarn and/or non-sealing agent material covering or sleeve may be in some embodiments, water soluble or biodegradable.

The scrim and/or the fabric, fiber, yarn and/or non-sealing agent material covering or sleeve may, in some embodiments, comprise an at least partial covering of a water soluble layer or film to at least partially cover and therefore protect that portion of the scrim and/or fabric covering, fiber, yarn and/or non-sealing agent material or sleeve, and/or any protect and/or prevent premature loss of any substances that may be coated on, adhered to, or seeded on or within the scrim and/or fabric covering, fiber, yarn and/or non-sealing agent material or sleeve. Moreover, such a water soluble film may be used to create a time-release structure or framework for exposure of the scrim to the blood and/or any substances that may be coated on, adhere to, or seeded on or within the scrim and/or fabric, fiber, yarn and/or non-scaling agent material covering or sleeve. FIG. 7 provides a cross-sectional view of an exemplary tubular graft 10 comprising an exemplary circumferential scrim 50 embedded within or adhered to the outer sealing layer 18 and a water soluble film 60 disposed over at least the scrim 50. It is to be understood that the scrim 50 may comprise substances as described herein that may be coated on the scrim 50, adhered to the scrim 50 and/or seeded on or within the scrim 50.

Self-Sealing and Hemostasis Achievement and/or Maintenance

Certain embodiments of the prosthetics, implants, such as, without limitation, tubular or non-tubular grafts, patches, sheets or tapes as described herein may comprise improvements that assist in achieving and/or maintaining hemostasis.

With continued reference to the Figures, sealing layer 18 (tubular exemplar) or 32 (non-tubular exemplar) may comprise a sealing agent formed of polymer, a co-polymer, a dielectric material, or an organic or non-organic polymer or co-polymer, any one of which may be combined in some embodiments with a foaming agent to improve self-sealing functionality of the graft. The sealing agents of the present invention are selected to provide one or more of, inter alia: imperviousness to electrocautery damage; improved blood shedding; improved visibility in the body; improved resistance to adherence of material to the outer surface; improved resistance to bacteria adherence to the sealing agent's outer surface; seroma formation and/or infection between a surface of the graft and biological tissue in contact with that graft surface; improved strength in self-sealing following a puncture; and improved water leakage or water permeability.

A preferred sealing agent in the various embodiments of the present invention is a non-organic polymer, one preferred non-organic polymer is silicone which, when combined with a foaming agent, forms a silicone foam rubber, most preferably in some embodiments a closed-cell silicone foam rubber. The process of forming silicone foam rubber (closed cell) is well known to the skilled artisan. For example, azodicarbonide foaming agent for producing many types of polymeric and elastomeric closed-cell foams will be readily recognized by the skilled artisan. Alternatively, perfluoroelastomer (“PFE”), or variations of PFE, may be used in place of a non-organic polymer such as silicone as it provides excellent self-sealing characteristics when punctured or cut. Still more alternatively, a non-organic polymer may be layered in combination with PFE, or variations thereof. In one alternative embodiment, exemplary silicone may be layered over the underlying textile layer with a layer, or regions, of PFE or variations thereof overlaying at least part of the underlayer of silicone. Still more alternatively, the underlayer disposed over the textile layer may be PFE and which may be covered by a layer of the exemplary silicone.

Some embodiments providing improvement in self-sealing due to puncture, e.g., suture hole punctures, discussed herein may be achieved using organic or inorganic polymers or co-polymers.

Generally, the self-sealing improvement is provided by the characteristics of the sealing layer. Sealing layer may comprise, in whole or in part, an organic or inorganic polymer or co-polymer that, when combined with a foaming agent as described above, may produce an open-cell structure or a closed-cell structure formed from the resulting organic or inorganic polymer, or co-polymer foam rubber.

Sealing layer may comprise a tissue-facing layer that comprises one or both of the open-cell or closed-cell structure. Sealing layer 18 may, at the interfacing surface, comprise an inorganic or organic polymer or co-polymer scaling agent. In this embodiment, the sealing agent covering and/or adhering to the interfacing surface is a polymer or co-polymer and may be organic or inorganic, but is not combined with a foaming agent. A closed-cell and/or open-cell foam rubber formed from an organic or inorganic polymer or co-polymer may be disposed or layered over the sealing agent to form the sealing layer 18.

Any combination of the closed-cell and/or open-cell construction may be used in combination with the inorganic or organic polymer or co-polymer sealing agent.

In some embodiments, the tissue-facing surface 20 (tubular), 33 (non-tubular) may comprise one, or both, of the open-cell and closed-cell organic or inorganic polymer or co-polymer foam rubber.

In some embodiments, the preferred polymer or co-polymer is inorganic and in some embodiments, a preferred inorganic polymer may comprise silicone. When used in combination with a polymer or co-polymer foam rubber as described herein, self-scaling of a graft puncture, e.g., a suture hole, occurs within one or more of: 10 minutes or less; 5 minutes or less; 2 minutes or less; 1 minute or less; 30 seconds or less, and/or 10 seconds or less.

The sealing layer configurations described above provide an improved closure of punctures through the sealing layer and/or textile layer, with an improved time of return to hemostasis of an implanted graft. For example, and without limitation, a self-sealing layer comprising a polymer or co-polymer foam rubber may enable a return to hemostasis following a graft puncture, e.g., self-scaling of a suture hole within one or more of: 10 minutes or less; 5 minutes or less; 2 minutes or less; 1 minute or less; 30 seconds or less, and/or 10 seconds or less.

Similarly, a scaling layer configuration comprising, e.g., an inorganic scaling agent layer or region within the sealing layer 18 wherein the sealing layer further comprises a layer of a closed-cell foam rubber as described herein may enable a return to hemostasis following a graft puncture, e.g., self-sealing of a suture hole within one or more of: 10 minutes or less; 5 minutes or less; 2 minutes or less; 1 minute or less; 30 seconds or less, and/or 10 seconds or less.

A sealing layer configuration comprising an organic or inorganic polymer or co-polymer sealing agent layer covering and/or adhering to the interfacing surface of the textile layer, wherein the sealing agent layer is covered, or integrated with, a closed-cell foam rubber as described herein may enable a return to hemostasis following a graft puncture, e.g., self-scaling of a suture hole within one or more of: 10 minutes or less; 5 minutes or less; 2 minutes or less; 1 minute or less; 30 seconds or less, and/or 10 seconds or less.

A scaling configuration comprising a layer of a closed-cell foam rubber as described herein and that covers and is adhered to the interfacing surface, wherein the layer of closed-cell foam rubber is covered by an organic or inorganic polymer or co-polymer scaling agent layer covering and/or adhering to the interfacing surface of the textile layer, wherein the scaling agent layer is covered, or integrated with, may enable a return to hemostasis following a graft puncture, e.g., self-scaling of a suture hole within one or more of: 10 minutes or less; 5 minutes or less; 2 minutes or less; 1 minute or less; 30 seconds or less, and/or 10 seconds or less.

In some embodiments, a hydrogel may be provided along portion(s) of the graft, wherein the hydrogel is configured to expand upon exposure to water and/or blood to block any bleeding that may occur. Accordingly, hydrogel may be embedded in the graft at points understood to be susceptible to bleeding. The hydrogel may be in some embodiments embedded between the fabric and the outer sealing layer, within the fabric, along the outer surface of the sealing layer, or any point between the fabric and the sealing layer.

In some embodiments, a sleeve may be provided where anastomosis may take place. In other embodiments, the number of suture holes may be minimized, e.g., 1 suture hole/mm, or 1 suture hole/2-3 mm.

### Infection Prevention or Mitigation

Certain embodiments of the implants or prosthetics, e.g., without limitation, tubular or non-tubular grafts, patches, sheets and/or tapes described herein may be configured to prevent and/or mitigate infection. Techniques for preventing or mitigating adherence of bacteria or other materials to the tissue-facing surface of embodiments of the graft are described above and which may aid in preventing or mitigating an infection from forming on the tissue-facing surface.

With reference to the Figures, additional infection prevention and mitigation structures are also now described.

Generally, an infection prevention or mitigation mechanism may be provided on the various implant or prosthetic embodiments described herein and may comprise (1) an applied infection prevention or mitigation mechanism; and/or (2) an embedded or impregnated infection prevention or mitigation mechanism.

The infection prevention or mitigation mechanism may comprise one or more, or at least one, antibiotic or antibiotic compound. When present, the at least one antibiotic compound may be applied just before implanting to the outer or tissue-facing surface of the subject tubular or non-tubular graft. Application of the at least one antibiotic compound may comprise spraying, brushing, soaking or other equivalent application and/or coating methods to provide a coating of the at least one antibiotic on the tissue-facing surface and/or may be applied via a scrim 50 or fabric sleeve 52 as described above.

The at least one antibiotic or antibiotic compound may be combined with one or more materials to form a composition which may be applied to the tissue-facing surface as described above. Such composition may comprise a carrier or other material that provides an enhanced stickiness or adherence to the tissue-facing surface so that, even with the super hydrophobic or antiwetting tissue-facing surfaces of some embodiments, the tissue-facing surface is ensured of having an at least partial layer of the antibiotic-laden composition. Moreover, the at least one antibiotic may be provided in a microencapsulated form wherein the capsule enables sticking to the sealing layer at the tissue-facing surface. Alternatively, the microencapsulated antibiotic may be held in place by being at least partially embedded within and/or pressed into the sealing layer at the tissue-facing surface. The microencapsulation is preferably biodegradable and/or water soluble to enable breakdown of the capsule to allow the encapsulated antibiotic to be released. The breakdown of the encapsulated antibiotic may be controlled in a timed release method by providing a range of capsule thickness, biodegradability, water solubility and/or materials whereby some of the capsules break down sooner and other capsules take longer time to break down.

Still more alternatively, the at least one antibiotic may be at least partially in a granular, powder or other non-liquid form which may be configured to adhere to, or be partially embedded into or pressed into, the sealing layer at the tissue-facing surface.

Other infection prevention and/or mitigation mechanisms may comprise impregnating the sealing layer with at least one antibiotic compound or composition and/or embedding the at least one antibiotic compound or composition within the sealing layer.

In some cases, the sealing layer may, e.g., at the interfacing surface, comprise at least a partial layer of an open cell foam rubber as described herein. The at least one antibiotic compound or composition thereof may then be disposed into at least some of the open cells along or near the interfacing surface. The antibiotic compound or composition disposed in the open cells may be liquid, non-liquid, encapsulated or not encapsulated, and may be time release enabled as described herein.

In some embodiments, the infection prevention and/or mitigation mechanism is configured to provide antibiotic material to prevent or mitigate any infections between the tissue-interfacing surface and the host tissue.

### Antibiotic Agents

As discussed, various embodiments of the present invention may further comprise an effective amount of at least one antibiotic or antibiotic compound coated along and/or embedded and/or incorporated within the outer sealing layer, or along other regions or portions, of a tubular or non-tubular graft. The at least one antibiotic may be encapsulated and/or non-encapsulated, or a combination of encapsulated and non-encapsulated and may be applied to or within a scrim 50 or fabric sleeve 52 as described supra.

The degradability or solubility of any capsule around the antibiotic may be modified to create a timed-release of the encapsulated antibiotic, wherein some capsules dissolve or degrade faster than others. Still further, some antibiotic may not be encapsulated and therefore fully available upon implantation. Moreover, a biodegradable or water soluble film layer may be provided over the antibiotic, whether or not encapsulated, to provide an additional mechanism for modifying the release of antibiotic over time.

Exemplary antibiotics for use in embodiments of the present invention may comprise mupirocin, defensin, gentamycin, geneticin, cefminoxime, penicillin, streptomycin, xylitol, rampafin or other antibiotic.

### Therapeutic Cells

“Therapeutic cells(s)” is defined herein to comprise at least one cell or type of cell, for example and without limitation a neural stem cell, that is applied to, embedded within, or otherwise incorporated within, a tubular or non-tubular graft of the present invention.

The therapeutic cell(s) may be derived from any source and may be at various stages of developmental differentiation as long as the therapeutic cell(s) are sufficient to stimulate growth of tissue into designated portions of the graft, either along a fabric or textile surface such as a scrim 50 or fabric sleeve 52 as described above, or along portions of the outer surface of a sealing layer. Moreover, it is recognized that the therapeutic cell(s) may be either heterologous or autologous to the host. By heterologous it is intended that the therapeutic cell is derived from a mammal other than the patient subject, while an autologous therapeutic cell is derived from the patient subject, manipulated ex vivo, and transported back into the patient by implanting grafts of the present invention.

As used herein, “regulatory agent” refers to any molecule having a growth, proliferative, differentiative, or trophic effect on an implanted therapeutic cell of the present invention. Any regulatory agent that is capable of regulating the development of the transplanted donor cell can be administered by the methods of the present invention. See, for example, Mackay-Sim et al. (2000) Prog. Neurobiol. 62:527-559, herein incorporated by reference. Further discussion of regulatory agent(s) is undertaken infra, each such aspect is included in the definition of “regulatory agent”.

As used herein, the terms “differentiate” and “mature” refer to the progression of a cell from a stage of having the potential to differentiate into at least two different cellular lineages to becoming a specialized cell. Such terms can be used interchangeably for the purposes of the present invention. The term “lineage” refers to all of the stages of the developmental cell type, from the earliest precursor cell to a completely mature cell (i.e., a specialized cell). Accordingly, the implanted therapeutic cells of the present invention can be derived from a multipotent cell lineage, preferably a neural lineage, and may be in any stage of differentiation. Thus, the present invention includes therapeutic cells that are naturally programmed to differentiate into only one type of lineage. These types of cells can include some kinds of fibroblasts or simply differentiated astroglia, neurons, oligodendrocytes, microglia or endothelial cells, and they may be derived or just isolated from the tissue of a donor or from amniotic tissue.

As used herein, the term “multipotent stem cell” refers to a cell capable of differentiating into a variety of lineages. Multipotent therapeutic, e.g., stem, cells are characterized by their ability to undergo continuous cellular proliferation, to regenerate exact copies of themselves (self-renewal), to generate a large number of regional cellular progeny, and to elaborate new cells in response to injury or disease. A “multipotent population of cells” refers to a composition of cells capable of differentiating into less than all lineages of cells but at least into two cell lineages. Current studies have demonstrated that multipotent stem cells from a non-neurologic region are not lineage-restricted to their developmental origin, but can generate region-specific neurons when exposed to the appropriate environmental cues (Lamga et al. (2001) J. Neurosci. 20:8727-8735).

The therapeutic cell(s) of the present invention can be derived from any fetal, amniotic or adult mammalian tissues, including bone marrow, or neural tissues. Moreover, the therapeutic cell(s) may include, but are not limited to, multipotent stem cells, progenitor cells, genetically engineered cells, t-cells and/or autologous cells.

Methods of isolation and transplantation of various progenitor cells derived from different tissues at different developmental stages are known in the art and include, for example, striatum cortex (Winkler et al. (1998) Mol. Cell. Neurosci. 11:99-116; Hammang et al. (1997) Exp. Neural. 147:84-95); cortex (Brustle et al. (1998) Nat. Biotechnol 16:1040-1044 and Sabate et al. (1995) Nat. Genet 9:256-260); human telencephalon (Flax et al (1998) Nature 392:18-24 and Vescovi et al, (1999) Neuron 11:951-966); hippocampus (Gage et al. (1995) J. Neurobiol. 36:249-266 and Suhonen et al. (1996) Nature 383:624-627); basal forebrain (Minger et al. (1996) Exp. Neurol. 141:12-24); ventral mesencephalon (Winkler et al. (1998) Mol. Cell. Neurosci. 11:99-116; Svendsen et al. (1996) Exp. Neural 137:376-388; Hammang et al. (1997) Exp. Neurol. 147:84-95; Studer et al. (1997) Nat. Neurosci. 1:290-295; Milward et al. (1997) J. Neurosci. Res. 50:862-871); and subventricular zone (Milward et al. (1997) Milward et al. (1997) J. Neurosci. Res. 50:862-871). Each of these references is herein incorporated by reference.

In addition, methods for the isolation of stem cell progeny and method to promote their differentiation can also be found in U.S. Pat. Nos. 6,071,889 and 6,103,530, both of which are herein incorporated by reference.

Therapeutic cells of the present invention may be altered to enhance tissue growth stimulation. For example, a preferred cell type is a human foreskin fibroblast, which is easily obtained and cultured (see, for example, U.S. Pat. No. 6,060,048). Such cells are preferably genetically altered, using methods known in the art, to express growth factors, neurotransmitters, neuropeptides, or enzymes involved in metabolism or tissue generation. See, for example, Gage et al. (1987) Neurosci. 23:795-807; Rosenberg et al. (1988) Science 242:1575-1578; Shimohama et al. (1989) Mol. Brain Res. 5:271-278; which are hereby incorporated by reference. Alternatively, therapeutic cells derived from a non-neuronal origin, such as epidermal cells, may be converted or transdifferentiated into different types of therapeutic cells. See, for example, U.S. Pat. No. 6,087,168.

As discussed, the therapeutic cell(s) of the present invention may be genetically altered prior to implantation into the host tissue with the graft structure. As used herein, the term “genetically altered” refers to a cell into which a foreign nucleic acid, e.g., DNA, has been introduced. The foreign nucleic acid may be introduced by a variety of techniques, including, but not limited to, calcium-phosphate-mediated transfection, DEAE-mediated transfection, microinjection, viral transformation, protoplast fusion, and lipofection. The genetically altered cell may express the foreign nucleic acid in either a transient or long-term manner. In general, transient expression occurs when foreign DNA does not stably integrate into the chromosomal DNA of the transfected cell. In contrast, long-term expression of foreign DNA occurs when the foreign DNA has been stably integrated into the chromosomal DNA of the transfected cell.

Such genes of interest include neurotransmitter-synthesizing enzymes (i.e., tyrosine hydrolase (TH) and cholineacetyltransferase). Such methods are commonly known in the art. For instance, therapeutic donor cells from various regions of the brain and at different stages of development have been isolated and have been immortalized via genetic alteration. For example, olfactory and cerebellum cells have been immortalized using the viral myc (v-myc) oncogene to generate cell lines with neuronal and glial phenotypes (Ryder et al. (1990) J. Neurobiol. 21:356). Similar studies by Snyder et al. ((1992) Cell 68:33) resulted in multipotent neuronal cell lines that were engrafted into the rat cerebellum to form neurons and glial cells. In other studies, murine neuroepithelial cells were immortalized with a retrovirus vector containing c-myc and were cultured with growth factors to form differentiated cell types similar to astrocytes and neurons (Barlett et al. (1988) Proc. Natl. Acad. Sci. USA 85:3255).

### Immunosuppressive Agents

Alternate embodiments of the present invention may further comprise an effective amount of at least one immunosuppressive agent to enhance the viability of the graft that does, or does not, comprise therapeutic cell(s) through protection from inflammatory response and/or activation of host immunocompetent cells. The immunosuppressive agents may be applied along, or embedded or incorporated within the graft and/or applied to or otherwise incorporated into a scrim 50 or fabric, fiber and/or yarn and/or non-scaling agent material comprising a sleeve as described above.

When immunocompetent cells of the host tissue detect an implanted graft of the present invention, inflammatory response and/or activation of host immunocompetent cells may result. This series of events may also decrease the therapeutic cell(s) survival, when present along or within an implanted graft. Therefore, immunosuppression agent(s) may be coated on and/or embedded or incorporated in a graft to play a crucial role in the survival and viability of not only the implanted graft itself, but also any therapeutic cells coated on or incorporated within an implanted graft. Conventional and well known immunosuppressive agents that may be used alone, or in combination, in the present invention comprise cyclosporine A, tacrolimus, prednisolone, azathioprine, methylprednisolone, mycophenylate mophetil and sirolimus. Another immunosuppressive agent comprises application of genetically engineered cells expressing the Fas ligand.

### Regulatory Agents

Certain regulatory agents to regulate, inter alia, growth and differentiation of the delivered therapeutic cells when present along or within an implanted exemplary prosthetic or implant such as a graft, patch, sheet and/or tape are within the scope of the present invention and include, for example, an effective amount of regulatory agents that promote the survival of the donor cells by modulating the immune and inflammatory response. Such regulatory agents include, for example, cyclosporin and various other immunomodulators, including, interleukins (i.e., IL-1, IL-2, IL-3, IL-4, IL-5, IL-6, IL-7, IL-8, IL-9, IL-10); tumor necrosis factors (i.e., TNF-alpha and TNF-beta); and, interferons (i.e., IFN-alpha, IFN-beta, IFN-gamma, IFN-omega, and IFN-tau); and any biologically active variants thereof. Further details regarding the administration of these immunomodulating agents by the methods of the present invention can be found in U.S. patent Ser. No. 09/733,168, entitled “Methods for Administering a Cytokine to the Central Nervous System and the Lymphatic System,” filed on Dec. 9, 2000, herein incorporated by reference.

Additional regulatory agents that find use in the methods of the invention include CAP23, a major cortical cytoskeleton-associated and calmodulin binding protein, and GAP43, a neural growth-associated protein. See, for example, Frey et al. (2000) J. Cell. Biol. 7:1443-1453. Further agents of interest include Osteogenic Protein-1 (OP-1) which is a morphogenic protein that stimulates growth, differention, and differentiation maintenance (U.S. Pat. No. 6,153,583); sonic hedgehog, a polypeptide shown to promote the survival of dopaminergic neurons (Miao et al. (1996) Cell Transplant 55:2-17); various other glial growth factors (U.S. Pat. Nos. 5,716,930; 6,147,190; and 5,530,109); and any biologically active variants thereof. All of these references are herein incorporated by reference.

Other regulatory agents of interest and within the scope of the present invention comprise growth factors. As used herein “growth factor” refers to a polypeptide capable of regulating the development of a therapeutic cell on or within an implanted graft. Growth factors useful in the methods of the present invention include, but are not limited to, members of the neurotrophin family (i.e., nerve growth factor (NGF), brain-derived neurotrophic factor (BDNF), neurotrophin-3 (NT-3), and neurotrophin-4 (NT-4, also known as NT-4/5 or NT-5); fibroblast growth factors (FGFs, i.e., basic fibroblast growth factor); epidermal growth factor family (i.e., EGF, TGF.alpha., amphiregulin, heparin-binding EGF-like growth factor (HB-EGF), batacelluin (BTC), and the neuregulin group); platelet-derived growth factor; insulin; insulin-like growth factors (i.e., IGF-I and IGF-2); ciliary neurotrophic factor (CNTF), glia cell line-derived neurotrophic factor family (GDNF) (i.e., GDNF and neurturin (NTN), persephin (PSP), and artemin (ART)); transforming growth factor .beta. superfamily (i.e., subfamilies include TGF beta 1, TGF beta 2, TGF beta 3, TGF beta 4, TGF beta 5, activin, inhibin, decapentaplegic); growth differentiation factors (GDF) (i.e., GDF1, GDF2, GDF3, GDF5, GDF6, GDF7, GDF8, GDF9, GDF9B, GDF10, GDF11, and GDF15); glia-derived nexin; activity dependent neurotrophic factor (ADNF); glial growth factor (GGF); and the like. It is further recognized that any biologically active variant of these growth factors is also useful in the methods of the present invention.

The regulatory agent of present invention may be from any animal species including, but not limited to, rodent, avian, canine, bovine, porcine, equine, and, preferably, human. Preferably the regulatory agent administered is from the same species as the animal undergoing treatment.

Biologically active variants of regulatory polypeptides (i.e., growth factors, such as IGF-I, NGF, and basic FGF, cytokines, etc.) are also encompassed by the various methods and pharmaceutical compositions of the present invention. Such variants should retain the biological activity of the regulatory agent, particularly the ability to regulate the development of the donor cell (i.e., promote the survival, maintain the desired phenotype, and/or regulate the developmental cues produced by the donor cell). For example, when the regulatory polypeptide is a growth factor, such as IGF-I, NGF-I, or a member of the FGF family, the ability to bind their respective receptor sites will be retained. Such receptor binding activity may be measured using standard bioassays.

One such regulatory agent, a growth factor, that is useful in the present invention is IGF-I. The term “IGF-I” as used herein refers to insulin-like growth factor I (IGF-I), a single-chain peptide having 70 amino acids and a molecular weight of about 7,600 daltons. Insulin-like growth factor I stimulates mitosis and growth processes associated with cell development. The amino acid and nucleotide sequence for IGF-I is known in the art. See, for example, U.S. Pat. No. 5,324,639 which discloses the human IGF-I sequence; Genbank Accession No. X15726, which discloses the sequence of bovine IGF-I; and Genbank Accession No. X06043 which discloses the sequence of rat IGF-I. Each of these references is herein incorporated by reference.

In another embodiment of the present invention, the regulatory agent may comprise a member of the FGF family of growth factors and/or biologically active variants thereof. The fibroblast growth factor family encompasses a group of structurally related proteins that bind heparin with a high affinity. FGF family members have mitogen activity and induce the proliferation of a wide variety of cell types. FGF family members also participate in angiogenesis, differentiation, cell migration, embryo development, and neuronal maintenance/survival. The term “FGF” as used herein refers to a member of the fibroblast growth factor family including, for example, FGF-1 (acidic FGF), FGF-2 (basic FGF), FGF-3, FGF-4, FGF-5, FGF-6, FGF-8, FGF-9, FGF-98, or a biologically active fragment or variant thereof. The amino acid sequence and methods for making many of the FGF family members are well known in the art.

In another embodiment of the present invention, the regulatory agent may comprise nerve growth factor (NGF) or a biologically active variant thereof. NGF was originally isolated as a complex having a molecular weight of 130 kDa and a sedimentation coefficient of 7S. This 7S complex included three types of subunits, with the “.beta.” subunit carrying all of the biological activities of NGF. Nerve growth factor stimulates mitosis and growth processes of cells, particularly nerve cells, and regulates development (i.e., influences repair, survival, and differentiation). The preferred amino acid sequence for human pre-pro-NGF and human mature NGF are provided in U.S. Pat. No. 5,288,622, which is incorporated herein by reference.

The NGF used in the present invention may be in its substantially purified, native, recombinantly produced form or in a chemically synthesized form. For example, the NGF can be isolated directly from cells naturally expressing NGF. NGF may also be recombinantly produced in eukaryotic or prokaryotic cell expression systems as described in Edwards et al, (1988) Mol. Cell. Biol. 8:2456; U.S. Pat. Nos. 5,986,070; and 6,005,081; all of which are herein incorporated by reference. Alternatively, the regulatory agent of the present invention may comprise erythropoietin (EPO), brain-derived neurotrophic factor (BDNF) and epidermal growth factor (EGF). Each of the regulatory agents described herein play a crucial role in the in-vivo survival and differentiation of the therapeutic cells of the present inventive methods and pharmaceutical compositions.

Administration of an effective amount of at least one regulatory agent by the methods of the present invention, i.e., coated along or incorporated or embedded within the sealing layer or other region of an implanted prosthetic, implant such as a graft, patch, sheet and/or tape and further including but not limited to incorporation within a scrim 50 or fabric, fiber or yarn or other non-sealing agent material sleeve 52, in combination with the therapeutic cells, will regulate development of the therapeutic cell of the graft. The phrase “regulate development” is intended herein to mean, inter alia, that the regulatory agent potentiates the survival, differentiation, axonal development, dendritic development, and/or proliferation of the transported therapeutic cell; improves adhesion of the transported therapeutic cells to surrounding tissues (i.e., incorporation into parenchymal tissue); improves the capacity of the transported therapeutic cells to establish synaptic connection with the host neurons (Le, enhances nerve fiber formation in the donor cells; increases nerve fiber projection distances of the donor cells; or enhances nerve fiber destiny of the donor cells); and/or instructs the transported therapeutic cell to commit to a specific neural lineage (i.e., adopt a neuronal (GABA-ergic neurons, dopaminergic neurons, cholinergic neurons, hippocampal neurons, and the like), astrocytic or oligodendritic cell fate). It is further recognized that a regulatory agent can potentiate the survival of a transplanted donor cell by modulating the immune response of the subject. By “modulate” is intended the down regulation of the immune or inflammatory response (i.e., influencing systemic immune function, antigen presentation, cytokine production, lymphocyte proliferation, and entry of lymphocytes and macrophages).

Furthermore, administration of the regulatory agent is known to “regulate development” of the an implanted therapeutic donor cell by influencing the developmental cues released by the transplanted cells (i.e., promote a donor cell to release neurotransmitters such as, dopamine, acetylcholine, GABA, or other neuroprotective factors). As such, the function and repair (i.e., enhanced nerve fiber formation, nerve fiber projection distances, and/or nerve fiber density) of the surrounding host tissue can be enhanced by the noninvasive methods of the present invention.

### Pharmaceutical Composition

A pharmaceutical composition may be applied to or incorporated within a tubular or non-tubular prosthetic or implant, e.g., and without limitation a graft, patch, sheet or tape and which may comprise one or more of an antibiotic, a therapeutic cell, a regulatory agent and/or an immunosuppressive agent. Such a pharmaceutical composition may comprise, in addition to the effective amount of at least one therapeutic cell, for example, at least one regulatory agent as described supra, at least one delivery-enhancement agent as described supra, at least one antibiotic, and/or at least one immunosuppressive agent, all as described herein. In addition, all of these substances, whether applied or incorporated to or within a graft as a composition or as individual substances may be encapsulated as described herein, and/or may be coated or covered with a degradable or water soluble film. In all cases, a timed-release of the various substances may be achieved by means described herein.

Further, any of the antibiotic(s), therapeutic cells, regulatory agent(s) and/or immunosuppressive agent(s) and/or the pharmaceutical composition of any of the listed substances may comprise any pharmaceutically acceptable additive, carrier, and/or adjuvant that may promote or enable the substance to be retained along or within the graft, but also to readily release the substance from the graft at a designed point in time.

By “pharmaceutically acceptable carrier” is intended a carrier that is conventionally used in the art to facilitate the storage, administration, and/or the biological activity of therapeutic cell(s), regulatory agent(s), antibiotic(s) and/or immunosuppressive agent within a pharmaceutical composition of the present invention. A carrier may also reduce any undesirable side effects of the components of such a pharmaceutical composition. A suitable carrier should be stable, i.e., incapable of reacting with other ingredients in the formulation. It should not produce significant local or systemic adverse effect in recipients at the dosages and concentrations employed for treatment. Such carriers are generally known in the art.

Suitable carriers for the various embodiments of the present invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like. A further pharmaceutical composition may comprise microparticles, organic and inorganic compounds serving as an adherence material for the cell(s) and cell conglomerates, or other substances such as antibiotics or immunosuppressive agents to the graft itself. These compounds may include several kinds of adhesive molecules, gels (serving as an encapsulating/embedding material for the cells), components of extracellular matrix or matrices, and organic and/or inorganic particles such as fibrin or fibronectin carbon- or clay- and dextran particles and their composition.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like. Where the carrier is a liquid, it is preferred that the carrier be hypotonic or isotonic with body fluids and have a pH within the range of 4.5-8.5.

Other acceptable components in the pharmaceutical composition comprise, without limitation, isotonicity-modifying agents such as water, saline, and buffers including phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein-based compositions, particularly pharmaceutical compositions, is well known in the art. See, Wang et al. (1980) J. Parent. Drug Assn, 34 (6): 452-462; Wang et al. (1988) J. Parent. Sci. Tech. 42: S4-S26 (Supplement); Lachman et al. (1968) Drug and Cosmetic Industry 102 (1): 36-38, 40, and 146-148; Akers (1988) J. Parent. Sci. Tech. 36 (5): 222-228; and Methods in Enzymology, Vol. XXV, ed. Colowick and Kaplan, “Reduction of Disulfide Bonds in Proteins with Dithiothreitol,” by Konigsberg, pp. 185-188.

In addition, some embodiments of an exemplary prosthetic or implant described herein such as a tubular or non-tubular graft, patch, sheet or tape may comprise a sealing layer with silver or colloidal silver which is a known antimicrobial, wherein the silver or colloidal silver is embedded, doped within and/or coated on the sealing layer. Sealing layers may comprise silicone with silver or colloidal silver.

In some embodiments, a layered approach to reducing seromas and/or infections with an implanted tubular or non-tubular graft may be employed. For example, an inner, or outer, coating of a carrier and an antibiotic operatively attached to the carrier e.g., may be provided.

Radiopaque and/or Visible Markings

Certain embodiments of the tubular or non-tubular prosthetics and implants such as grafts, patches, sheets and tapes described herein may comprise radiopaque marking that aid the medical practitioner to, using the aid of imaging instruments and techniques, locate the position of the graft, ensure that the location is appropriate, evaluate the condition of the implanted graft, and/or help prevent occlusion of branch arteries and veins. A non-limiting list of imaging instruments and/or techniques includes x-ray, fluoroscopy, CT scan, and MRI.

Visual markings may also be provided to help the medical practitioner determine whether there is a twist or other malformation of the subject graft and may represent the length or other dimension of the graft under physiological pressures. For example, markings that indicate 1 cm length increments would be measured at 1 cm at physiological pressures and may be measured at greater or less than 1 cm in length at other than physiological pressures. As defined herein, visual markings are markings that are visible to the naked eye of the practitioner and enhanced only by magnifying glasses and without aid of imaging equipment or other enhanced techniques.

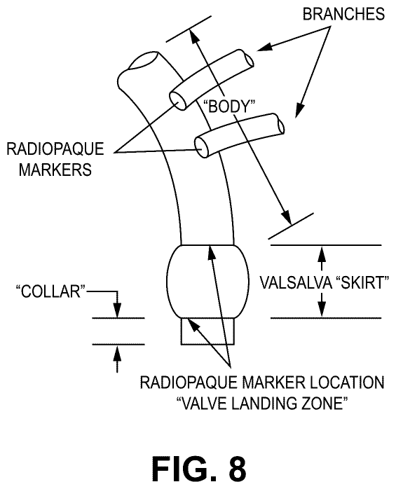

With reference now to FIG. 8 and with continued reference to FIGS. 1-7, the radiopaque markings may comprise one or more regions of radiopacity that is/are disposed at one or more locations along the graft. One or more of the regions of radiopacity may be located at the interfacing surface of the textile layer, i.e., beneath the sealing agent layer 18. Other regions of radiopacity may be embedded within the sealing layer 18 without structural connection with the interfacing surface of the textile layer 18.

FIG. 8 illustrates exemplary radiopaque markers incorporated into a Valsalva graft as rings at the sino-tubular junction and the aortic root for use as “landing zones” for future fluoroscopy guided valve procedures. A first radiopaque marker ring is shown located between the collar and Vasalva skirt and a second radiopaque marker ring positioned at the upper end of the Valsalva skirt. Radiopaque markers may be used in other tubular or non-tubular grafts described herein. For example, and without limitation, radiopaque material or markers may comprise a complete or partial or intermittent ring around a branch location(s), e.g., native vessels or grafts, to enable identification during fluoroscopy-guided interventions. See FIG. 8 for exemplary branch locations for radiopaque markers or rings.

In some embodiments, the radiopaque regions or markings may be visible to the naked eye of the medical practitioner without imaging or equipment or other vision enhancing aid. In this embodiment, the visible radiopaque regions may also serve as the visual markings described above.

In other embodiments, the radiopaque regions or markings are not visible to the naked eye of the medical practitioner and must be visualized using imaging techniques.

Visual markings, as described above, help to quickly assess whether there is a twist or other malformation of the subject graft and may represent the length or other dimension of the graft under physiological pressures. For example, markings that indicate 1 cm length increments would be measured at 1 cm at physiological pressures and may be measured at greater or less than 1 cm in length at other than physiological pressures.

In an exemplary tubular graft, two or more visual markings may be provided at, or near, the tissue-facing surface of the sealing layer. In certain embodiments, the two or more visual markings may be aligned with a non-deformed longitudinal axis of the tubular graft and may be radially spaced apart from each other. In certain embodiments, the two or more radially spaced-apart visual markings may be visually different from each other to help the practitioner assess, among other things, whether the graft is twisted. For example, the markings may differ in color, or may be unbroken vs broken or dashed, discrete points vs dashes or unbroken to provide a visually differentiating characteristic between the markings.

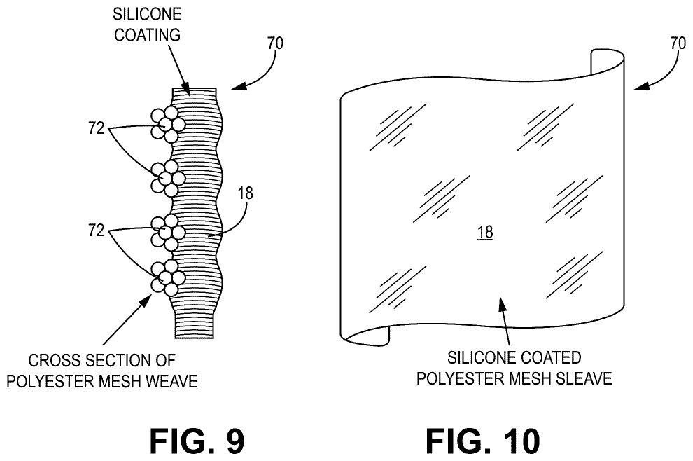

Various embodiments of the tubular or non-tubular grafts described herein may be used as dissection planes. Generally, the surface of the graft comprising woven textile enables superior anchoring to the tissue it is adjacent to and, when the woven textile is, on the opposing surface, completely covered with a non-organic polymer or co-polymer such as silicone, no tissue is allowed to grow into that surface. See, e.g., FIGS. 1-3C. A preferred material and design is shown in FIGS. 9 and 10, comprising an exemplary non-tubular graft 70 with outer sealing layer 18 forming a complete cover over an embedded polyester mesh weave material 72 which is partially exposed on the opposing surface. A portion of the polyester mesh weave material 72 is embedded within, or covered by, the outer sealing layer 18 which may comprise an organic or inorganic polymer or co-polymer. A preferred polymer comprises silicone, but other polymers may be used so long as no tissue ingrowth is allowed or enabled through, or into, the outer sealing layer material. In some cases, as shown in FIG. 9, pockets of exemplary silicone may be exposed on the tissue-facing surface such that tissue ingrowth occurs in a pattern that corresponds with the exposed polyester mesh weave. In other embodiments, the exemplary silicone is prevented from reaching the tissue-facing surface and the entire tissue-facing surface is, therefore, available for tissue ingrowth. The opposing surface of the outer sealing layer 18, however, is not available for tissue ingrowth which is useful to provide a surgical dissection plane for surgical procedures following implantation. As noted, such a surgical dissection plane may be provided in both tubular and non-tubular grafts as those are described herein.

The surgical dissection plane embodiments may be used in, without limitation, the following.

Wrapping ventricular assist devices (“VAD”), including right and left ventricular assist devices.
Heart valve leaflets.
Heart valve bases or commissures.
Extracorporeal membrane oxygenation (“ECMO”.
Guided tissue regeneration.
Venous surgery.
Rotator cuff buttresses.
Knee joint pads.
Leg ligaments.
Cranial pads.
Dental implants.
Rhinoplasty.
Burns.
Heart valve reinforcement.

Selected inventive embodiments follow:
1. A tubular or non-tubular implant or prosthetic configured to ease explantation from a living body, comprising:
   a woven textile layer comprising a plurality of interstices and/or pores, and defining an interfacing surface and an opposing surface; and
   a sealing layer of non-porous, non-organic polymer adhered to the interfacing surface and defining a tissue-facing surface, wherein the non-porous, non-organic polymer is configured to partially penetrate through at least some of the plurality of interstices and/or pores at the opposing surface of the woven textile layer,
   wherein the non-porous, non-organic polymer of the sealing layer does not penetrate to the opposing surface of the woven textile layer, and
   wherein the non-porous, non-organic polymer of the sealing layer comprises a resistance to heat that is greater than a resistance to heat of organic polymers or organic co-polymers, whereby the outer layer is configured to resist and/or retard damage from applied electrosurgical energy,
   whereby the inner textile layer is protected, and damage thereto is resisted and/or retarded, from the applied electrosurgical energy.
2. The tubular or non-tubular implant or prosthetic of claim 1, wherein the non-porous, non-organic polymer of the sealing layer comprises molecular bonds that comprise a binding energy that is higher than the binding energy of carbon bonds.
3. The tubular or non-tubular implant or prosthetic of claim 1, wherein the sealing layer is hydrophobic.
4. The tubular or non-tubular implant or prosthetic of claim 1, wherein the sealing layer is super-hydrophobic and comprises a sliding angle of <10 degrees.
5. The tubular or non-tubular implant or prosthetic of claim, wherein the sealing layer is super anti-wetting, comprises a sliding angle of <10 degrees and sheds blood therefrom.
6. The tubular or non-tubular implant or prosthetic of claim 1, wherein the sealing layer comprises no interstices or pores therethrough.
7. The tubular or non-tubular implant or prosthetic of claim 1, further comprising a water permeability value that is 0 mL/cm²/min.
8. The tubular or non-tubular implant or prosthetic of claim 1, wherein the tissue-facing surface of the outer layer comprises a surface roughness with a root mean square average of 35 nm or less, wherein the surface roughness of the tissue-facing surface is configured to prevent adhesion of bodily tissue or platelets to at least part of the tissue-facing surface following implantation of the tubular implant or prosthetic.
9. The tubular or non-tubular implant or prosthetic of claim 1, wherein the sealing layer is configured to prevent biological tissue and/or platelet adhesion to at least part of the tissue-facing surface.
10. The tubular or non-tubular implant or prosthetic of claim 1, wherein the sealing layer is configured to prevent bacteria from adhering to the tissue-facing surface.
11. The tubular or non-tubular implant or prosthetic of claim 1, wherein the sealing layer is configured to prevent seromas from forming along the tissue-facing surface.
12. The tubular or non-tubular implant or prosthetic of claim 1, wherein the sealing layer is configured to prevent infection from forming along the tissue-facing surface.
13. The tubular or non-tubular implant or prosthetic of claim 1, wherein at least a part of the opposing surface of the textile layer allows tissue ingrowth.
14. The tubular or non-tubular implant or prosthetic of claim 1, wherein the sealing layer prevents transmural communication or migration from the luminal surface into the outer layer.
15. The tubular or non-tubular implant or prosthetic of claim 1, wherein at least a portion of the sealing layer comprises a color that contrasts with blood and tissue.
16. The tubular or non-tubular implant or prosthetic of claim 1, comprising three cross-sectional zones:
   zone 1, bounded on a first side by the opposing surface of the textile layer and wherein the plurality of interstices and/or pores do not comprise any of the non-porous, non-organic polymer of the sealing layer;
   zone 2, located within the textile layer and located adjacent to, and bounded on a first side, by zone 1 and on an opposing side by the interfacing surface, wherein at least some of the plurality of interstices and/or pores comprise the non-porous, non-organic polymer; and
   zone 3, located adjacent to, and bounded on an inner side, by the interfacing surface and bounded on an outer side by the tissue-facing surface defined by the sealing layer, wherein zone 3 consists only of the non-porous, non-organic polymer, wherein zone 3 defines a thickness.
17. The tubular or non-tubular implant or prosthetic of claim 16, wherein the non-porous, non-organic polymer of zone 3 prevents biological tissue ingrowth or transmural communication through the interfacing surface into zone 3.
18. The tubular or non-tubular implant or prosthetic of claim 17, wherein biological tissue ingrowth into zone 1 and at least a portion of zone 2 is not prevented.
19. The tubular or non-tubular implant or prosthetic of claim 1, wherein the non-porous, non-organic polymer comprises a foaming agent forming a polymer foam rubber.
20. The tubular or non-tubular implant or prosthetic of claim 19, wherein the polymer foam rubber forms a polymer foam rubber closed-cell structure.
21. The tubular or non-tubular implant or prosthetic of claim 19, wherein at least the tissue-facing surface of the sealing layer comprises one or both of an open-cell polymer foam rubber structure and a closed-cell polymer foam rubber structure.
22. The tubular or non-tubular implant or prosthetic of claim 1, wherein the graft is tubular and configured to be connectable to one or both of an inlet or an outlet of a left ventricular assist device or a right ventricular assist device.
23. The tubular or non-tubular implant or prosthetic of claim 1, wherein the graft is tubular and configured to comprise a Valsalva aortic root graft.
24. The tubular or non-tubular implant or prosthetic of claim 1, wherein the sealing layer provides protection and insulation against heat transfer and/or electrical energy to and/or through the textile layer during intravascular procedures.
25. The tubular or non-tubular implant or prosthetic of claim 24, wherein the sealing layer protects blood cells within or proximate to the woven textile from thermal or electrical damage.
26. The tubular or non-tubular implant or prosthetic of claim 24, wherein the sealing layer protects therapeutic cells, therapeutic agents and/or antibiotics or other substances adhered to or incorporated within the woven textile from thermal or electrical damage.
27. The tubular or non-tubular implant or prosthetic of claim 24, wherein the sealing layer protects tissue on or near the woven textile from thermal or electrical damage.
28. A tubular or non-tubular implant or prosthetic configured to ease explantation from a living body with controlled tissue ingrowth and bodily incorporation of the implanted graft, comprising:
   a textile layer comprising a plurality of interstices and/or pores, and defining an interfacing surface and an opposing surface; and
   a sealing layer of non-porous, non-organic polymer adhered to at least part of the interfacing surface and defining a tissue-facing surface, wherein the non-porous, non-organic polymer is configured to partially penetrate through at least some of the plurality of interstices and/or pores at the opposing surface of the textile layer,
   wherein the sealing layer is interrupted by at least one fabric scrim configured to facilitate tissue ingrowth therein.
29. The implant or prosthetic of claim 28, wherein the at least one fabric scrim is operatively connected with the textile layer at the interfacing surface.
30. The implant or prosthetic of claim 28, wherein the at least one fabric scrim is embedded within the sealing layer, wherein at least one fabric scrim is spaced from the textile layer by the non-porous, non-organic polymer of the sealing layer.
31. The implant or prosthetic of claim 28, wherein at least one of the group consisting of: antibiotics, therapeutic cells, regulatory agents, and immunosuppressive agents, is embedded and/or operatively connected with the at least one scrim.
32. The implant or prosthetic of claim 31, wherein at least one of the Markush group of claim 28 are encapsulated with a water soluble or biodegradable or bioerodable material.
33. The implant or prosthetic of claim 28, further comprising a water soluble, biodegradable or bioerodable film covering the at least one scrim.
34. The implant or prosthetic of claim 28, wherein at least a portion of the at least one scrim comprises a color that contrasts with blood and/or tissue.
35. The implant or prosthetic of claim 34, wherein at least a portion of the at least one scrim is a blue color.
36. A tubular or non-tubular implant or prosthetic configured for radiopacity, comprising: a textile layer comprising a plurality of interstices and/or pores, and defining an interfacing surface and an opposing surface;
   a sealing layer of non-porous, non-organic polymer adhered to at least part of the interfacing surface and defining a tissue-facing surface, wherein the non-porous, non-organic polymer is configured to partially penetrate through at least some of the plurality of interstices and/or pores at the opposing surface of the textile layer; and
   one or more regions of radiopacity comprising radiopaque material, wherein the radiopaque material is embedded within the sealing layer and/or attached to the interfacing surface of the textile layer.
37. The implant or prosthetic of claim 36, wherein the implant or prosthetic comprises a Valsalva graft and wherein the one or more regions of radiopacity comprise radiopaque rings configured to be located at a connection to the aortic root and/or the sino-tubular junction.
38. The implant or prosthetic of claim 36, wherein the radiopaque rings are complete rings or partial, intermittent rings.
39. The implant or prosthetic of claim 36, wherein the one or more regions of radiopacity are also visible to the eye of the practitioner and configured to enable assessment and diagnosis of the position and condition of the graft.
40. A tubular or non-tubular implant or prosthetic, comprising:
   a textile layer comprising: a plurality of interstices and/or pores, and defining an interfacing surface and an opposing surface;
   a sealing layer of non-porous, non-organic polymer adhered to at least part of the interfacing surface and defining a tissue-facing surface, wherein the non-porous, non-organic polymer is configured to partially penetrate through at least some of the plurality of interstices and/or pores at the opposing surface of the textile layer; and
   one or more regions of exposed textile layer within the sealing layer, wherein the one or more regions of exposed textile layer are not covered by the sealing layer and are at least partially surrounded by the sealing layer.
41. The implant or prosthetic of claim 40, wherein the one or more regions of exposed textile layer are configured to allow tissue ingrowth.
42. The implant or prosthetic of claim 41, wherein the one or more regions of exposed textile layer are configured for use as dissection planes.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

The invention claimed is:

1-25. (canceled)

26. A tubular or non-tubular implant or prosthetic comprising:
a textile layer comprising a plurality of interstices and/or pores, and defining an interfacing surface and an opposing surface; and
a sealing layer comprising a non-porous, non-organic polymer adhered to the interfacing surface and defining a tissue-facing surface, wherein the non-porous, non-organic polymer is configured to at least partially penetrate through at least some of the plurality of interstices and/or pores at the interfacing surface of the textile layer,
wherein the non-porous, non-organic polymer is formed of a material configured to prevent electrocautery energy applied thereto from damaging the textile layer.

27. The tubular or non-tubular implant or prosthetic of claim 26, wherein the material of the non-porous, non-organic polymer of the sealing layer is impervious to electrocautery energy applied thereto.

28. The tubular or non-tubular implant or prosthetic of claim 26, wherein the material of the non-porous, non-organic polymer of the sealing layer is configured to prevent electrocautery energy applied thereto from damaging the textile layer when an electrocautery device applying the electrocautery energy is operated at any power or voltage level.

29. The tubular or non-tubular implant or prosthetic of claim 26, wherein the material comprises silicone.

30. The tubular or non-tubular implant or prosthetic of claim 26, wherein the material comprises silicone foam rubber.

31. The tubular or non-tubular implant or prosthetic of claim 26, wherein the material defines a molecular bond energy higher than 355 KJ/mol.

32. The tubular or non-tubular implant or prosthetic of claim 26, wherein the material of the non-porous, non-organic polymer of the sealing layer is configured to resist heat transfer to the textile layer from heat applied to the sealing layer.

33. The tubular or non-tubular implant or prosthetic of claim 26 comprising a cross-section formed of at least a first zone, a second zone, and a third zone,
wherein the first zone is within the textile layer and is bounded on a first side by the opposing surface of the textile layer and wherein the plurality of interstices and/or pores within the first zone do not comprise any of the non-porous, non-organic polymer of the sealing layer,
wherein the second zone is within the textile layer and located adjacent to and bounded on a first side by the first zone and on an opposing side by the interfacing surface, wherein the plurality of interstices and/or pores within the second zone comprise at least some of the non-porous, non-organic polymer,
wherein the third zone is within the sealing layer and is located adjacent to and bounded on a first side by the interfacing surface of the textile layer and on an opposing side by the tissue-facing surface of the sealing layer, and
wherein the material of the non-porous, non-organic polymer is configured to prevent the electrocautery energy applied to the tissue-facing surface of the sealing layer from damaging at least the first zone.

34. The tubular or non-tubular implant or prosthetic of claim 33, wherein the material of the non-porous, non-organic polymer is configured to prevent the electrocautery energy applied to the tissue-facing surface of the sealing layer from damaging at least the first zone and the second zone.

35. The tubular or non-tubular implant or prosthetic of claim 26, wherein the non-porous, non-organic polymer of the sealing layer does not penetrate to the opposing surface of the textile layer.

36. The tubular or non-tubular implant or prosthetic of claim 26, wherein at least some of the non-porous, non-organic polymer of the sealing layer penetrates to the opposing surface of the textile layer.

37. The tubular or non-tubular implant or prosthetic of claim 26, wherein the material of the non-porous, non-organic polymer is configured to prevent bacteria from adhering to the tissue-facing surface.

38. The tubular or non-tubular implant or prosthetic of claim 26, wherein the material of the non-porous, non-organic polymer is configured to prevent seromas from forming on the tissue-facing surface.

39. The tubular or non-tubular implant or prosthetic of claim 26, wherein the material of the non-porous, non-organic polymer is configured to prevent infection from forming on the tissue-facing surface.

40. A tubular or non-tubular implant or prosthetic comprising:
a textile layer comprising a plurality of interstices and/or pores, and defining an interfacing surface and an opposing surface; and
a sealing layer comprising a non-porous, non-organic polymer attached to the interfacing surface and defining a tissue-facing surface, wherein the non-porous, non-organic polymer is configured to at least partially penetrate through at least some of the plurality of interstices and/or pores at the interfacing surface of the textile layer,
wherein the non-porous, non-organic polymer is formed of a material configured to prevent electrocautery energy applied to or near the tissue-facing surface from damaging the textile layer.

41. The tubular or non-tubular implant or prosthetic of claim 40, wherein the material of the non-porous, non-organic polymer of the sealing layer is impervious to electrocautery energy applied thereto.

42. The tubular or non-tubular implant or prosthetic of claim 40, wherein the material of the non-porous, non-organic polymer of the sealing layer is configured to prevent electrocautery energy applied to or near the tissue-facing surface from damaging the textile layer when an electrocautery device applying the electrocautery energy is operated at any power or voltage level.

43. The tubular or non-tubular implant or prosthetic of claim 40, wherein the material comprises silicone.

44. The tubular or non-tubular implant or prosthetic of claim 40, wherein the material comprises silicone foam rubber.

45. The tubular or non-tubular implant or prosthetic of claim 40, wherein the material defines a molecular bond energy higher than 355 KJ/mol.

46. A tubular or non-tubular implant or prosthetic configured to resist or retard damage due to application of electrocautery energy, comprising:
a textile layer comprising a plurality of interstices and/or pores, and defining an interfacing surface and an opposing surface; and
a sealing layer of a non-porous, non-organic polymer attached to the interfacing surface and defining a tissue-facing surface, wherein the non-porous, non-organic polymer is configured to at least partially penetrate through at least some of the plurality of interstices and/or pores at the interfacing surface of the textile layer,
wherein the non-porous, non-organic polymer of the sealing layer does not penetrate to the opposing surface of the textile layer,
wherein the sealing layer is configured to resist and/or retard damage from applied electrosurgical energy,
whereby damage to the textile layer resulting from the applied electrosurgical energy is resisted and/or retarded.
